(19) United States
(12) Patent Application Publication
(10) Pub. No.: US 2026/0273078 A1
(43) Pub. Date: Sep. 17, 2026

(54) AN INJECTABLE DELIVERY SYSTEM FOR LONG-ACTING ADMINISTRATION OF DRUGS

(71) Applicant: QUEEN'S UNIVERSITY OF BELFAST, Belfast Antrim (GB)

(72) Inventors: Garry LAVERTY, Draperstown (GB); Sreekanth PENTLAVALLI, Belfast (GB); Sophie COULTER, Bangor, Down (GB); Emily CROSS, St Helens, Merseyside (GB)

(73) Assignee: QUEEN'S UNIVERSITY OF BELFAST, Belfast Antrim (GB)

(21) Appl. No.: 19/469,322

(22) PCT Filed: Mar. 27, 2024
(86) PCT No.: PCT/EP2024/058389

§ 371 Date: Sep. 25, 2025

(30) Foreign Application Priority Data
Mar. 31, 2023 (GB) 2304871.3

(51) Int. Cl. A61K 47/64; A61K 9/00; C07K 7/06

(52) U.S. Cl. CPC A61K 47/64; A61K 9/0024; C07K 7/06

(57) ABSTRACT

The invention relates to an injectable delivery system for long-acting administration of drugs. The invention includes a compound formed at least partially from a peptide-mimetic molecule. The compound is configured for the attachment of one or more drug molecules, and is further configured to release the one or more drug molecules under physiological conditions. A solution of the compound forms a hydrogel in response to an enzyme that is endogenous to a human or animal. A solution of the compound can be administered to the skin, wherein upon administration it forms a hydrogel, and any drug attached to the compound is then gradually released.

### FIELD OF THE INVENTION

The invention relates to a compound that can be used in a solution as an injectable delivery system for long-acting administration of drugs.

### BACKGROUND

Injectable delivery systems for long-acting administration of drugs have potential uses for a wide range of treatments or as a preventative. One particular potential use is as a contraceptive. A further use is for protection against HIV/AIDS. The only options currently available for simultaneous protection against unintended pregnancy and HIV/AIDS are the male and female condoms. However, patient acceptability and overall effectiveness of condoms is invariably poor. Therefore, there is an urgent need for new practical combined HIV/AIDS-contraceptive products. A number of concepts are currently progressing through early stage clinical studies, most notably a dapivirine+levonorgestrel vaginal ring.

Despite the popularity of long-acting injectable contraceptives (e.g. Depo-Provera®) and the clinical progress of new long-acting injectable antiretroviral formulations (e.g. GSK/Janssen's injectable rilpivirine+cabotegravir: Cabenuva®), there are no reports to date describing long-acting injectable strategies for combined administration of both antiretroviral and contraceptive drugs. This is very challenging as both HIV antiretrovirals and contraceptive drugs are water insoluble and tend to interact and aggregate when mixed in water-based formulations such as suspensions.

Cabenuva® itself, recently licensed by the FDA (USA) and medicine regulators in Europe, Australia and Zimbabwe, is two separate injections of rilpivirine+cabotegravir, rather than one single administered combined product. Having a single injection would improve patient compliance to medicine, especially for those who are averse to needles, and improve storage and distribution.

Existing long-acting injectable technologies rely almost exclusively on pre-formed implants and suspension-type formulations, which suffer particular disadvantages that limit their use as single or combined HIV-contraceptive products. For example, pre-formed subdermal contraceptive implants comprising non-biodegradable polymers (e.g. Nexplanon®) require costly and painful surgical procedures for insertion and removal. This procedure is also prone to causing infection.

Drug suspensions (e.g. rilpivirine+cabotegravir) are fundamentally unstable, requiring formulation strategies to ensure that physical stability and drug particle size is maintained over the product shelf-life, particularly in settings with widely fluctuating daily temperatures and lack of cold chain supply e.g. sub-Saharan Africa where need for this product is most. Instability of the formulation leads to problems in administering the product to patients. Unstable/insoluble particles can become trapped in the syringe meaning not all the product can be administered effectively.

Also, these formulations commonly produce relatively high initial drug concentrations in the systemic compartment (the so-called burst effect), with drug concentrations declining steadily thereafter, making it difficult to optimise drug dosing and pharmacokinetics. This has a significant impact on drug treatment/prevention strategies.

Polymeric subdermal implants already exist, with several products currently marketed mainly in the contraceptive field. These include Jadelle®, Implanon®, Nexplanon®, and Probuphine®. The active pharmaceutical agents in these products are mostly steroids. Other long-acting products include Probuphine® implant, which contains the drug buprenorphine hydrochloride and is utilised for opioid addiction.

Long-acting, injectable treatments for psychiatric illness and drug dependence are also known. They include intramuscular administration of: i) aqueous suspensions (e.g. paliperidone: Xeplion®), ii) oil-based injections (e.g. haloperidol: Haldol®, fluphenazine: Modecate®), iii) microspheres (risperidone: risperdal Consta®, naltrexone: Vivitrol®). Alternative long-acting systems tend to rely on pre-formed polymeric implants (buprenorphine: Probuphine®), which have disadvantages that limit their wider use e.g. comprising non-biodegradable polymers that require painful and costly surgery for insertion/removal. Similarly, these approaches have significant limitations including: poorly tunable properties; high burst release of drug making drug dosing difficult to control; persistent pain (oily injections); and poor physical stability (suspensions). There is also little scope for adjusting their performance, e.g. to readily incorporate multiple drugs or future drug candidates.

Injectable drug delivery systems also have potential use in cancer treatments. Current long-acting delivery systems include OncoGel™, a controlled-release depot formulation of paclitaxel that utilises the ReGel™ platform. This forms a gel after injection at body temperature, being a low viscosity solution at room temperature. It is therefore an example of a thermosensitive controlled-release delivery system.

The present invention obviates or mitigates the problems outlined above with existing injectable products for long-acting administration of drugs. Specifically, the present invention seeks to provide a system that can be easily administered and may comprise multiple drugs in a single product. The present invention further obviates or mitigates the problems with the requirement to administer a high frequency of individual drug doses to treat diseases, and in particular to treat cancer and psychiatric illnesses. The present invention seeks to provide a new contraceptive and HIV-preventative drug delivery platform. The present invention further obviates or mitigates the problem of known long-acting drug formulations producing relatively high initial drug concentrations in the systemic compartment.

### SUMMARY OF THE INVENTION

According to a first aspect of the invention there is provided a compound formed at least partially from a peptide-mimetic molecule, wherein the compound is configured for the attachment of one or more drug molecules, wherein the compound is further configured to release the one or more drug molecules under physiological conditions, and wherein a solution of the compound forms a hydrogel in response to an enzyme that is endogenous to a human or animal.

Ideally, the endogenous enzyme is an enzyme that can usually be found within the skin and throughout the body.

The peptide-mimetic molecule may be any of one or more of the group comprising peptoids, D-peptides, or β-peptides (for example, β-homo peptides), —N-methyl peptides, cyclic peptides, or gamma peptides.

The compound is soluble and can be formulated as a solution for injectable administration. As a solution containing the compound forms a hydrogel in response to an endogenous enzyme, the solution will form a hydrogel upon administration under the skin of the human or animal recipient. The solution is capable of forming tissue-like hydrogels that can be tailored to gradually release drugs for at least 28, 42 days, or 49 days, or the required dosing interval. This will remove the need for patients to comply with complex drug dosing regimens on a daily basis and improve their adherence to medication.

Preferably, the compound comprises a peptide-mimetic backbone, formed from one or more of the group comprising peptoids, D-peptides, or β-homo peptides. Peptoids, D-peptides, or β-homo peptides are peptide-like molecules that, unlike peptides, are not rapidly broken down by proteases.

The enzyme that is endogenous to a human or animal may be phosphatase, an esterase, a protease, a matrix metalloproteinase, thrombin, trypsin e.g. chymotrypsin, β-galactosidase, lipase, transglutaminase, thermolysin, glucose oxidase, peroxidase or tyrosinase. Preferably, a solution of the compound forms a hydrogel in response to phosphatases. Advantageously, as phosphatases are present in skin and tissue, the solution forms a hydrogel upon administration to the skin or tissue. Ideally, the compound comprises a phosphate group which when cleaved drives formation of a hydrogel.

Preferably, the compound comprises a site for covalent attachment of a drug. The site for covalent attachment may be provided by an amino acid that has a primary amine (—NH₂) as its functional group or R-group that allows drug attachment. These include the amino acid arginine and the unnatural amino acid variants 2,4-diamino butyric acid, 2,3-diaminopropionic acid, ornithine, epsilon (ε) lysine. The unnatural forms differ to lysine by the length of their carbon/methylene chain (—CH₂) between the primary amine group (—NH₂) and the α-carbon.

Similarly various cysteine derivates can have varying lengths for the carbon/methylene chain (—CH₂) length. Cysteine has one methylene unit between thiol (—SH) group and the α-carbon while homocysteine has two methylene units. Penicillamine (d-isomer of dimethylcysteine) is an amino acid derivative that could potentially replace cysteine as it also contains a thiol group (—SH).

The site for covalent attachment of a drug may be an ε-amino group of lysine, or an L- or D-cysteine. This enables easy attachment of a drug to the compound for use as an injectable delivery system for long-acting administration of the drug.

Preferably, the one or more drugs are releasable from the compound by hydrolysis. Advantageously, the drug will be released from the compound under physiological conditions. The one or more drugs may be attached to the compound by an ester, carbamate, amide, ether, disulphide or hydrazone linkage.

The drug attachable or attached to the compound may be an HIV/AIDS antiretroviral drug, contraceptive, antipsychotic, or cancer treatment drug. The drug may be zidovudine, MIV-150, cabotegravir, lamivudine, etonogestrel, haloperidol, or doxorubicin.

The compound may comprise L-peptides. Switching D-peptides in the compound with L-peptides can increase the rate at which the compound degrades in the body.

The compound of the above aspect of the invention when attached to a drug may be used as a medicament. The compound may be configured for use in the prevention or treatment of AIDS, psychiatric illnesses, cancer, or as a contraceptive.

The compound may have the formula:

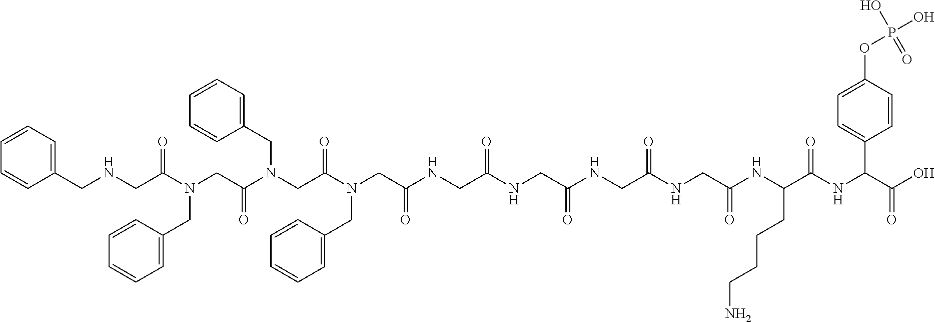 [SMILES: NCCCCC(NC(=O)CNC(=O)CNC(=O)CNC(=O)CNC(=O)CN(Cc1ccccc1)C(=O)CN(Cc1ccccc1)C(=O)CN(Cc1ccccc1)C(=O)CNCc1ccccc1)C(=O)NC(C(=O)O)c1ccc(OP(=O)(O)O)cc1]

Preferably, at least 10%, 20%, 30%, 40%, 50%, 60%, 70%, 80%, or 90% of a concentration of the compound, with or without a drug attached thereto, remains after 28 days of incubation with proteinase K. The compound is therefore biostable and is not easily broken down by proteases upon administration.

According to a second aspect of the invention there is provided an injectable delivery system for long-acting administration of drugs, the system comprising a solution, the solution comprising the compound of the first aspect of the invention, wherein the solution can be injected under the skin, and wherein upon injection and exposure to enzymes within or under the skin, the compound forms a hydrogel. The compound may be further attached to a drug.

This system exists as a soluble injection for improved ease of administration under the skin (subcutaneous or intramuscular) or to difficult to deliver sites across the body (eye, spine, tumour). The system forms a hydrogel implant in response to enzymes (phosphatases), present within the skin and throughout the body, to release drugs long-term and thereby removing the need for multiple daily dosing.

The injectable delivery system may be used as a medicament and may be configured for use in the prevention or treatment of AIDS, depression, cancer, or as a contraceptive.

The wherein the drug may be detectable in the recipient after at least 28, 42 or 49 days.

According to a third aspect of the invention there is provided a compound having the formula:

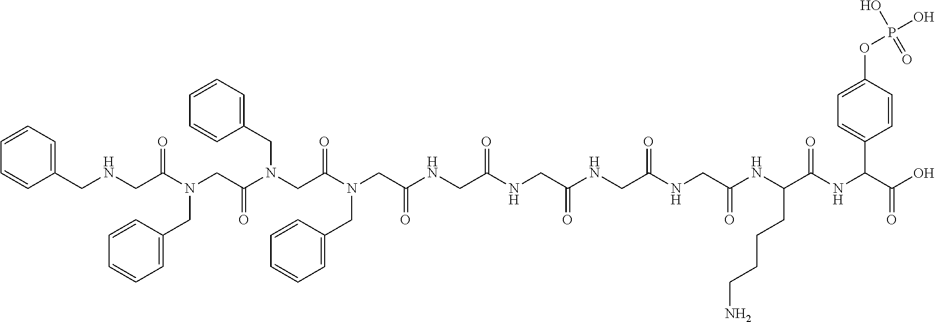 [SMILES: NCCCCC(NC(=O)CNC(=O)CNC(=O)CNC(=O)CNC(=O)CN(Cc1ccccc1)C(=O)CN(Cc1ccccc1)C(=O)CN(Cc1ccccc1)C(=O)CNCc1ccccc1)C(=O)NC(C(=O)O)c1ccc(OP(=O)(O)O)cc1]

### LIST OF FIGURES

Specific implementations of the present disclosure will now be described, by way of example only, and with reference to the accompanying drawings in which:

FIG. 1 shows the chemical structure of a) peptoid-peptide (PP4G), (NPhe)₄(Gly)₄(L/DLys)-(L/DTyr[H₂PO₄])—OH, which is a compound according to the present invention, and b) model drugs tested with the system of the present invention: HIV/AIDS antiretovirals (zidovudine, lamivudine, MIV-150, cabotegravir), contraceptive (etonogestrel), antipsychotic (haloperidol) and anticancer (doxorubicin).

FIG. 2 shows the synthetic route for conjugation of model drugs to PP4G, a compound according to the present invention. a i) tert-butyldimethylsilyl chloride (TBDMSCl), imidazole in anhydrous DMF (16 hours). a ii) 4,4′-dimethyoxytrityl chloride (DMTrCl) in anhydrous pyridine. a iii) tert-butylammonium fluoride (TBAF) in tetrahydrofuran (THF) (6 hours). b) succinic anhydride, 4-dimethyl-aminopyridine (DMAP), anhydrous pyridine (12 hours). c) N-hydroxysuccinimide (NHS), N,N′-diisopropylcarbodiimide (DIC) in chloroform (6 hours). Upon completion filter to remove N,N′-diisopropylurea (DIU). d i) Sodium hydrogen carbonate (NaHCO₃) [to pH 7.6], water, acetone (2 hours). d ii) Redissolve in trifluoroacetic acid (TFA), methanol, acetonitrile (overnight).

FIG. 3 shows the concept of enzyme responsive peptoid-peptide hydrogels, such as the compound of the present invention, for sustained release of drugs (e.g. using HIV NRTI antiretroviral lamivudine as displayed). i) formulation administered as soluble subcutaneous injection ii) removal of phosphate group by phosphatase enzymes results in rapid formation of hydrogel depot under the skin. iii) hydrolysis of ester bond, under physiological conditions, results in sustained release of chemically unmodified drug from hydrogel.

FIG. 4 shows rheological analysis of 5% w/v hydrogels of peptoid-peptides according to the invention containing 4 glycines (PP4G) and 5 glycines (PP5G) and no drug attached.

FIG. 5 shows rheological analysis of 5% w/v hydrogels of peptoid-peptides containing 4 glycines according to the invention (PP4G: D-peptides at ky amino acid positions) with HIV/AIDS drug cabotegravir covalently attached. Storage modulus data are represented by filled circles and loss modulus by non-filled circles.

FIG. 6 shows rheological analysis of 5% w/v hydrogels of peptoid-peptides containing 4 glycines according to the invention (PP4G: L-peptides at KY amino acid positions) with HIV/AIDS drug zidovudine covalently attached. Storage modulus data are represented by filled circles and loss modulus by non-filled circles.

FIG. 7 shows rheological analysis of 5% w/v hydrogels of peptoid-peptides containing 4 glycines according to the invention (PP4G: D-peptides at ky amino acid positions) with HIV/AIDS drug MIV-150 covalently attached. Storage modulus data are represented by filled circles and loss modulus by non-filled circles.

FIG. 8 shows rheological analysis of 5% w/v hydrogels of peptoid-peptides containing 4 glycines according to the invention (PP4G: D-peptides at ky amino acid positions) with contraceptive drug etonogestrel covalently attached.

FIG. 9 shows rheological analysis of 5% w/v hydrogels of peptoid-peptides containing 4 glycines according to the invention (PP4G: D-peptides at ky amino acid positions) with HIV/AIDS drug MIV-150 covalently attached and contraceptive etonogestrel also attached to a separate PP4G molecule. This are mixed and formulated to a 9:1 ratio (PP4G-MIV-150:PP4G-ENG) as etonogestrel is highly potent therefore it is present at a reduced ratio within this formulation mix.

FIG. 10 shows rheological analysis of 5% w/v hydrogels of peptoid-peptides containing 4 glycines according to the invention (PP4G: D-peptides at ky amino acid positions) with antipsychotic drug haloperidol covalently attached.

FIG. 11 shows the biostability of peptide and peptoid-peptide hydrogel forming molecules according to the invention after incubation with the broad-spectrum protease, proteinase K, over 28 days. L-peptide NapFFKY(p)G-OH (small circle) and D-peptide NapffkY(p)G-OH (upright triangle) are peptide-only controls that also form hydrogels in response to phosphatase enzymes.

FIG. 12 shows the cumulative release of zidovudine (AZT) from physically encapsulated and chemically bonded to the peptoid-peptide compound according to the invention and L and D-peptide controls in pH 7.4 Phosphate Buffered Saline over 28 days. Data relates to a) 28 days and b) the first 72 hours of drug release. A 30-60% reduction in drug burst release can be demonstrated for model drugs when chemically conjugated compared to physically mixed (see also FIGS. 13-16). Key: (NPhe)₄GGGGKY(p)-OH: peptoid-peptide with 4 glycines (PP4G) and L-enantiomers of amino acids lysine (K) and tyrosine (Y); NapFFKY(p)G-OH; a L-peptide hydrogel control; Napffky(p)G-OH: a D-peptide hydrogel control.

FIG. 13 shows the cumulative release of cabotegravir (CAB) from physically encapsulated and chemically bonded to the peptoid-peptide compound according to the invention in pH 7.4 Phosphate Buffered Saline over 28 days. Data relates to a) 28 days and b) the first 72 hours of drug release. Key: (NPhe)₄GGGGky(p)-OH: peptoid-peptide with 4 glycines (PP4G) and D-enantiomers of amino acids lysine (k) and tyrosine (y).

FIG. 14 shows the cumulative release of MIV-150 and etonogestrel (ENG) from physically encapsulated and chemically bonded to the peptoid-peptide compound according to the invention in pH 7.4 Phosphate Buffered Saline over 28 days. Data relates to a) 28 days and b) the first 72 hours of drug release. Key: (NPhe)₄GGGGky(p)-OH: peptoid-peptide with 4 glycines (PP4G) and D-enantiomers of amino acids lysine (k) and tyrosine (y).

FIG. 15 shows the cumulative release of haloperidol from physically encapsulated and chemically bonded to the peptoid-peptide compound according to the invention in pH 7.4 Phosphate Buffered Saline over 28 days. Data relates to a) 28 days and b) the first 72 hours of drug release.

FIG. 16 shows a) In vivo zidovudine (AZT) drug plasma concentrations obtained across 35 days in Sprague Dawley rats (n=6) subcutaneous administration of peptoid-peptide (PP4G), D-peptide containing variant. Subcutaneous administration of PP4G(AZT) in Sprague Dawley rats demonstrated zidovudine (AZT) blood plasma concentrations at least within the IC₅₀ range (30-130 ng/ML; represented by horizontal bars) for 35 days. b) Weight of Sprague Dawley rats (n=6) at the beginning (day 0) and end (day 35) of the experiment in the control, intravenous and subcutaneous administration groups.

FIG. 17 shows MIV-150 and Etonogestrel (ENG) plasma concentration in Sprague Dawley rats, with peptoid-D-peptide-drugs administered individually.

FIG. 18 shows MIV-150 and Etonogestrel (ENG) plasma concentration in Sprague Dawley rats, with Peptoid-D-peptide-drugs administered as combined product MIV-150: ENG (8:2 ratio).

### DETAILED DESCRIPTION

The formula of an example of a compound according to the present invention is

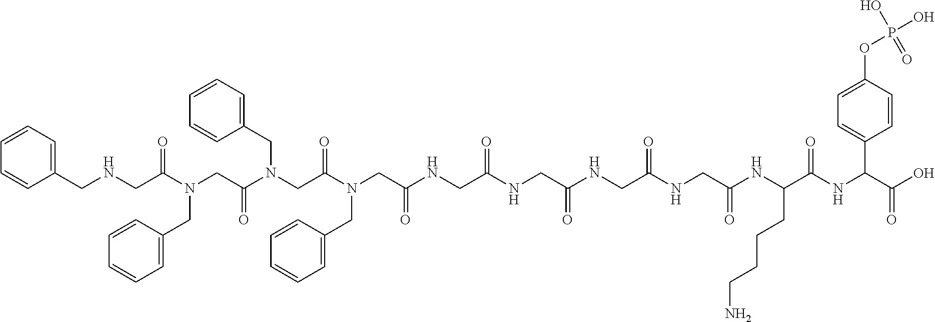 [SMILES: NCCCCC(NC(=O)CNC(=O)CNC(=O)CNC(=O)CNC(=O)CN(Cc1ccccc1)C(=O)CN(Cc1ccccc1)C(=O)CN(Cc1ccccc1)C(=O)CNCc1ccccc1)C(=O)NC(C(=O)O)c1ccc(OP(=O)(O)O)cc1]

The compound is a peptoid-peptide with four glycines, herein referred to as PP4G. The compound can form a hydrogel when in solution and when exposed to phosphatase enzymes.

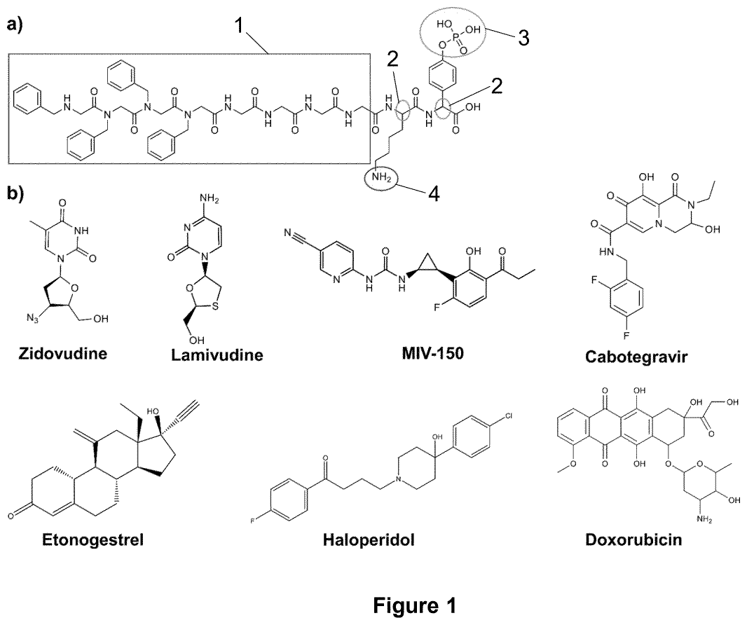

Referring to FIG. 1, structure a), which is the above compound, provides a hydrogel forming sequence 1 composed of aromatic functional groups which allows self-assembly into hydrogels due to intermolecular interactions e.g. IT-IT stacking of benzene rings, hydrogen bonding of amide groups. This is a peptide-mimetic backbone. In the embodiment provided above, the backbone is formed from peptoids and D-peptides, but alternative peptide-mimetic molecules can be used if desired.

L or D peptide sequences can be selected at lysine and/or tyrosine 2 to modify biostability/degradation for specific dosage intervals. A phosphate group 3 endows solubility when attached to a peptide-mimetic backbone. When this phosphate group is removed e.g. by phosphatase enzymes in the subcutaneous skin space, this acts as a physiological trigger to drive rapid hydrogel formation.

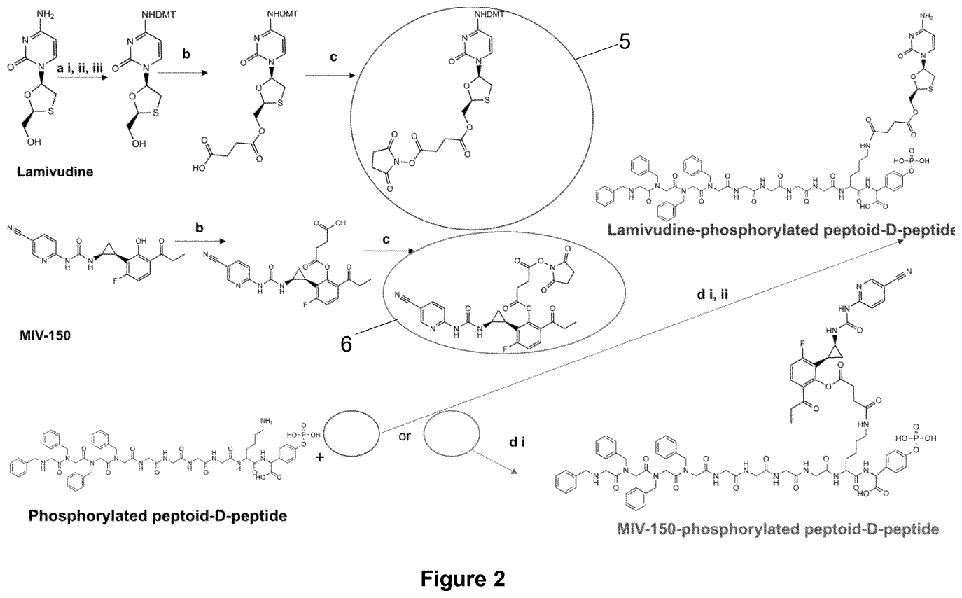

The compound further has a site for attachment of a drug molecule. The site for attachment of a drug molecule may be an s-amino group of lysine, or an L- or D-cysteine. Lamivudine, MIV-150 and cabotegravir can be covalently attached to an embodiment of the present invention via succinic acid (drug-succi-NHS active ester) to the s-amino group of lysine 4 by an ester-amide exchange reaction (outlined fully in FIG. 2). This ester-drug bond is readily hydrolysed under physiological conditions (pH 7.4, H₂O, esterase enzymes) to release unmodified drug from peptoid-peptide hydrogel. There is clinical rationale for combining different HIV drug classes such as these as they will work synergistically via separate modes of antiviral action. Etonogestrel (contraceptive drug) and haloperidol (antipsychotic) can also be added to peptoid-peptide covalently as it contains a single —OH group. Doxorubicin can be precisely conjugated to the sulphydryl group of a L or D-cysteine via a Michael addition reaction involving a maleimide-glycine linker. In this case, the L or D-lysine of peptoid-peptide is substituted with a L or D-cysteine to enable covalent drug attachment.

Referring now to FIG. 2, Lamivduine (shown) requires additional synthetic step (a) for protection of reactive amine group (—NH₂). MIV-150 (shown) and cabotegravir follow the same synthetic route and do not require this initial protection step. Antiretrovirals then follow similar synthetic steps (b-d) resulting in activation of succinic anhydride to protected lamivudine 5 and MIV-150 6. This creates a functional group that allows directed attachment (step d) to the terminal primary amide side group of the peptoid-peptide gelator forming an ester linkage.

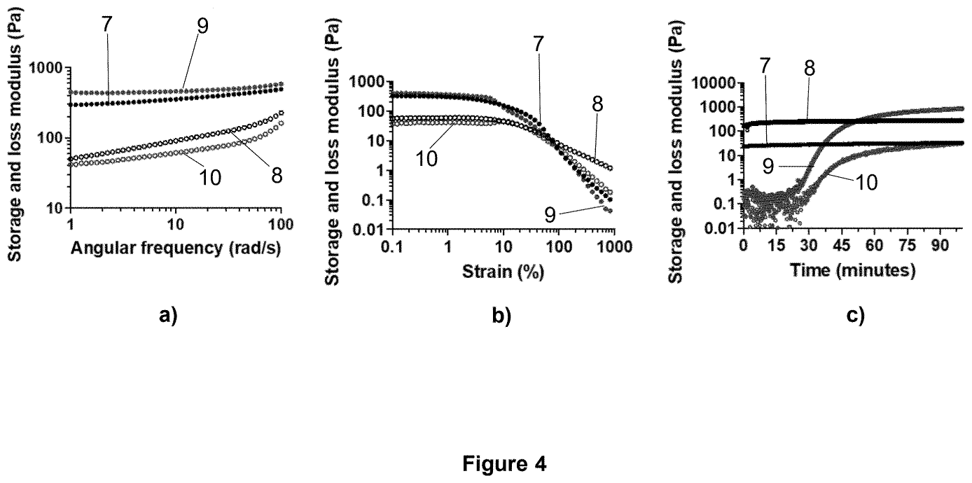

Referring now to FIG. 4, the graphs show PP4G storage modulus 7, PP4G loss modulus 8, PP5G storage modulus 9, and PP5G loss modulus 10. Gel stiffness defined by frequency sweep (a), and gel strength defined by strain sweep (b), are similar for both PP4G and PP5G. However, time for gelation in response to phosphatase enzymes is quicker for PP4G. This more beneficial to long-acting injectable application. Quick gel formation upon injection acts as an additional diffusional barrier to the entry and exit of water from the gel. Water causes breakage/hydrolysis of drug-peptoid/peptide bond, and transports drugs out of the hydrogel, therefore causing more rapid burst release of drug.

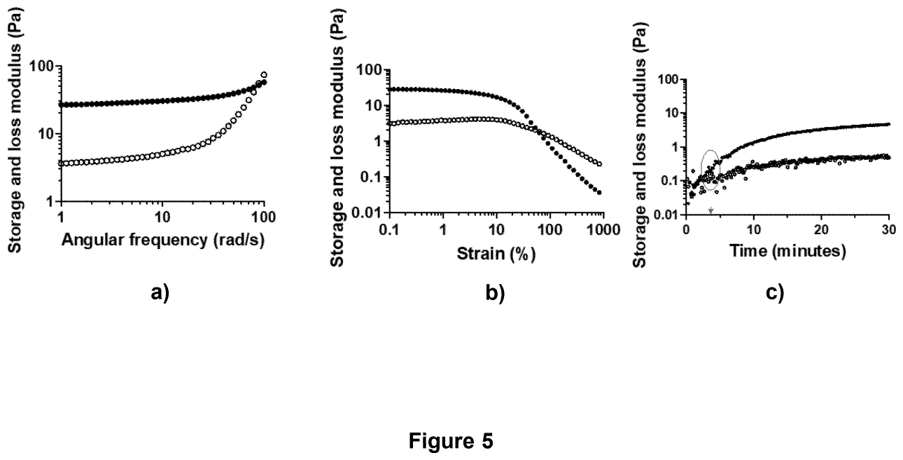

FIG. 5 shows rheology data for an embodiment of the invention wherein PP4G is covalently bonded cabotegravir. FIG. 5 a) shows that drug attachment results in a slight decrease in gel stiffness, compared to non-drug attached PP4G (see FIG. 4). b) Gel strength is similar in both cabotegravir attached and non-drug attached PP4G. c) PP4G cabotegravir covalently attached takes ~4 minutes to gel in the presence of phosphatase enzymes.

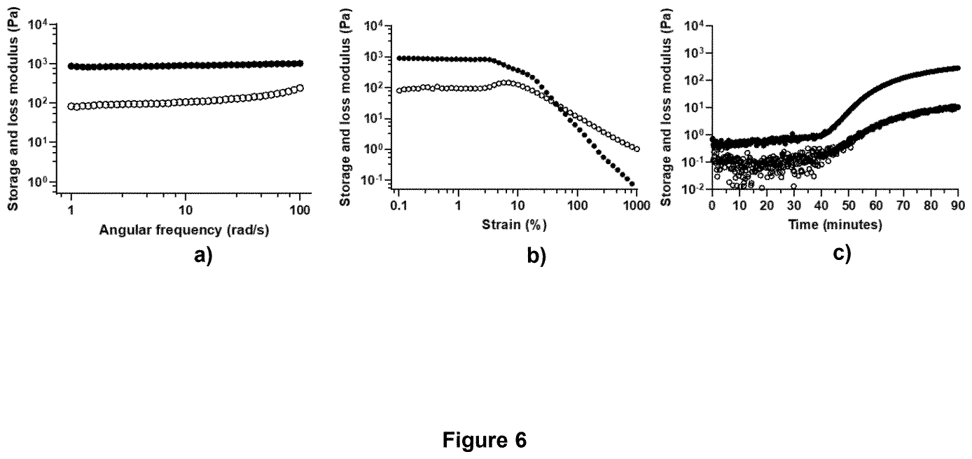

FIG. 6 shows rheology data for an embodiment of the invention wherein PP4G is covalently bonded to the HIV/AIDS drug zidovudine. FIG. 6 shows that similar gel stiffness (a) and strength (b) is demonstrated for PP4G with drug attached when compared to PP4G alone. c) Gel formation occurs ~45 minutes after exposure to phosphatase enzymes.

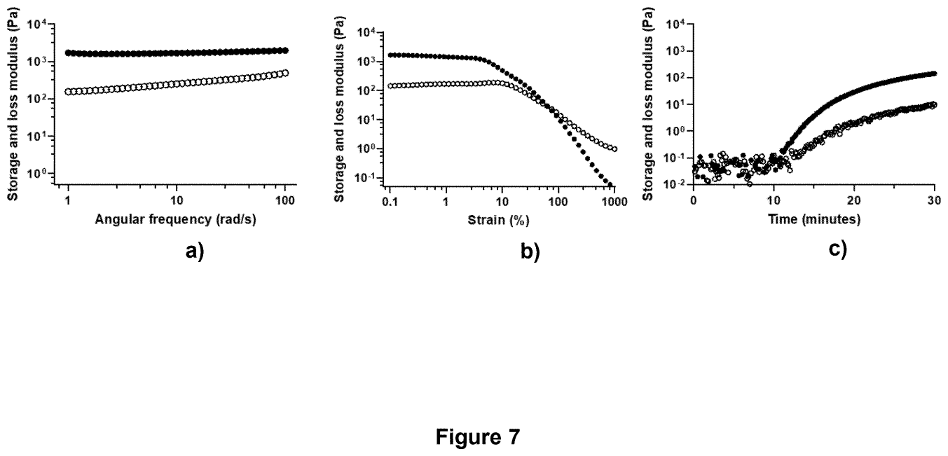

FIG. 7 shows rheology data for an embodiment of the invention wherein PP4G is covalently bonded HIV/AIDS drug MIV-150. FIG. 6 shows similar gel stiffness (a) and strength (b) is demonstrated compared to PP4G alone. c) Gel formation occurs ~12 minutes after exposure to phosphatase enzymes.

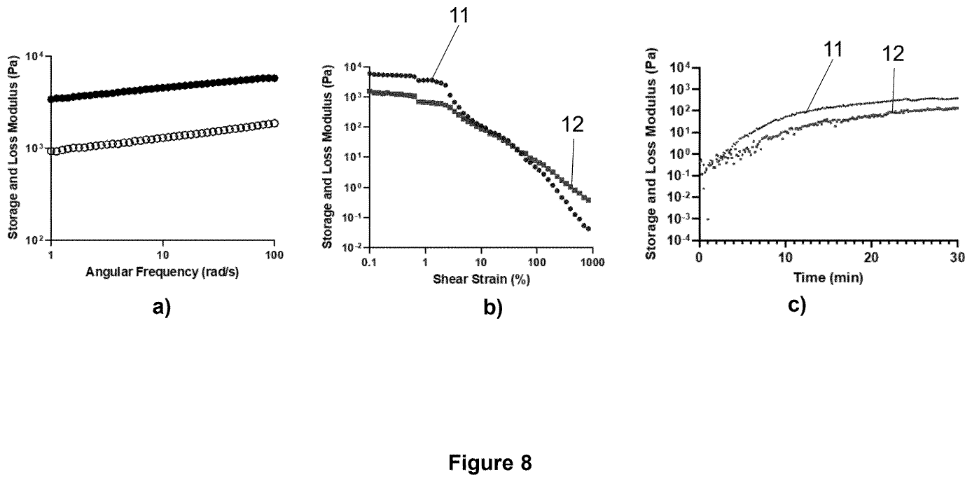

FIG. 8 shows rheological analysis of 5% w/v hydrogels of peptoid-peptides containing 4 glycines according to the invention (PP4G: D-peptides at ky amino acid positions) with contraceptive drug etonogestrel covalently attached. Similar gel stiffness (a) to PP4G alone; storage modulus data are represented by filled circles and loss modulus by non-filled circles. Gel strength (b) is slightly weaker with PP4G-ENG hydrogels compared to PP4G alone; storage modulus 11 and loss modulus 12 shown. c) Gel formation also occurs ~12 minutes after exposure to phosphatase enzymes; storage modulus 11 and loss modulus 12 shown.

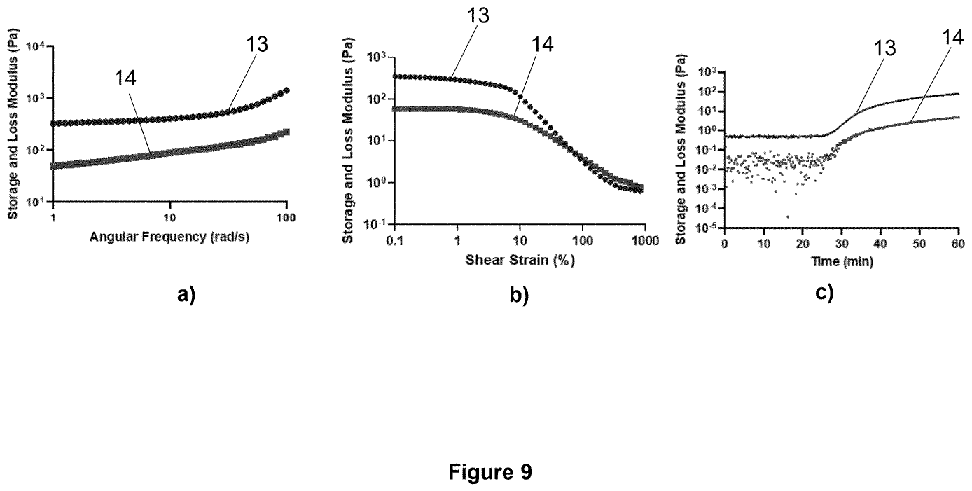

FIG. 9 shows rheological analysis of 5% w/v hydrogels of peptoid-peptides containing 4 glycines according to the invention (PP4G: D-peptides at ky amino acid positions) with HIV/AIDS drug MIV-150 covalently attached and contraceptive etonogestrel also attached to a separate PP4G molecule. This are mixed and formulated to a 9:1 ratio (PP4G-MIV-150:PP4G-ENG) as etonogestrel is highly potent therefore it is present at a reduced ratio within this formulation mix. Slightly weaker gel stiffness (a) and similar gel strength (b) is demonstrated compared to PP4G alone. c) Gel formation occurs ~30 minutes after exposure to phosphatase enzymes. Storage modulus 13, and loss modulus 14 shown.

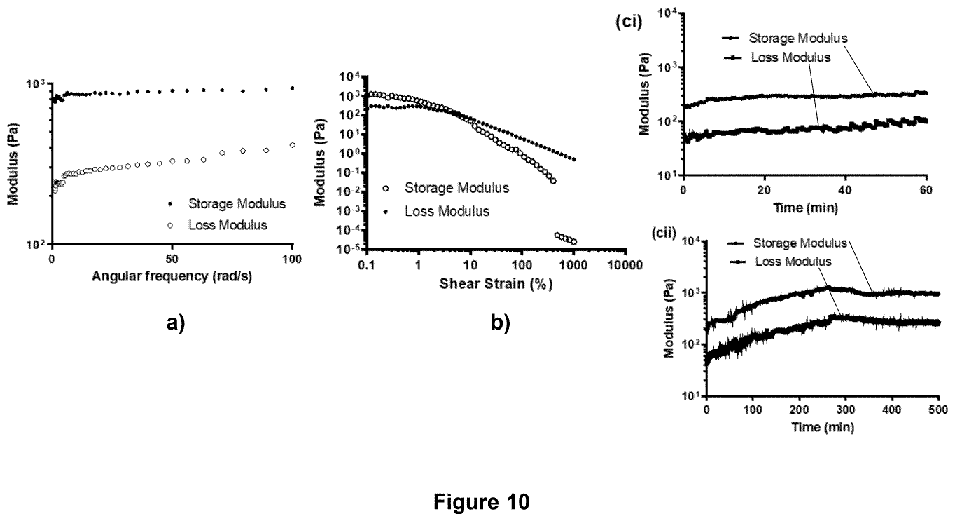

FIG. 10 shows rheological analysis of 5% w/v hydrogels of peptoid-peptides containing 4 glycines according to the invention (PP4G: D-peptides at ky amino acid positions) with antipsychotic drug haloperidol covalently attached. Similar gel stiffness (a) and strength (b) compared to PP4G alone. ci) Gel formation also occurs immediately after exposure to phosphatase enzymes with fully formed gel stiffness occurring after ~270 minutes.

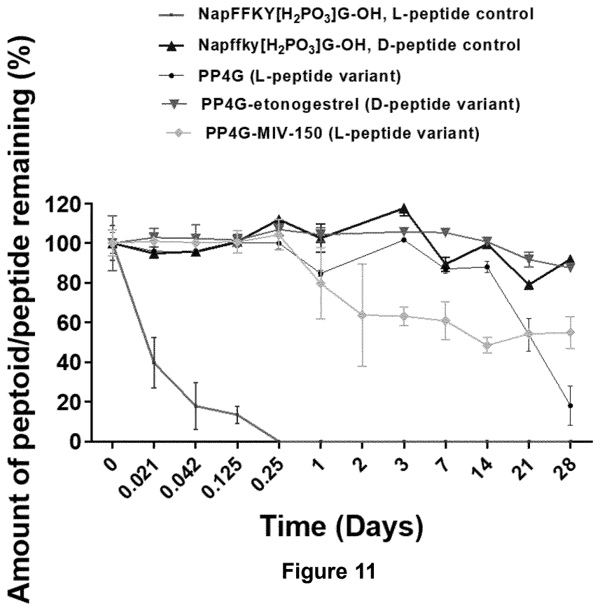

Referring now to FIG. 11, L-peptide degrades rapidly, within hours, in the presence of proteinase-K. D-peptides still show significant stability after 28 days exposure to proteinase-K. The L-peptide variant of peptoid-peptide PP4G and PP4G-MIV-150, where K (lysine) and Y (tyrosine) amino acid are L-enantiomers, breaks down between days of proteinase-K exposure. The D-peptide variant of peptoid-peptide PP4G, where k and y amino acid are unnatural D-enantiomers, remains significantly stable for 28 days exposure, wherein approximately 90% of the concentration of the compound remains after 28 days. Biostability can therefore be tailored, for example to break down over a particular dosage interval, in order to allow administration of next dose of PP4G+drug.

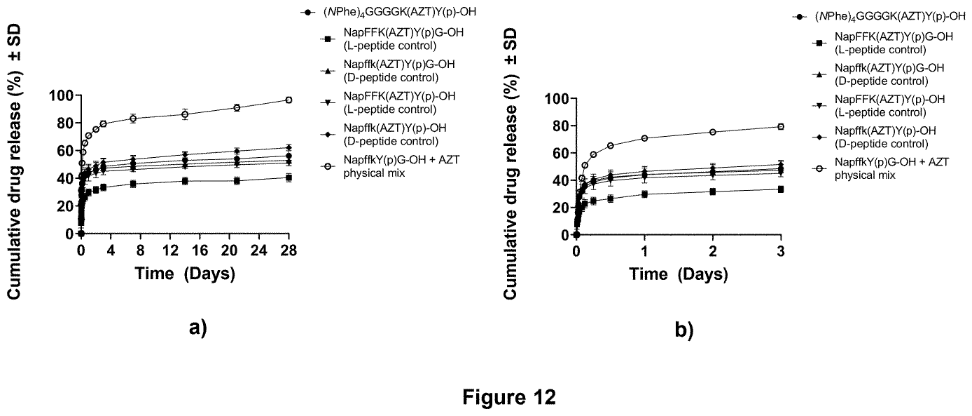

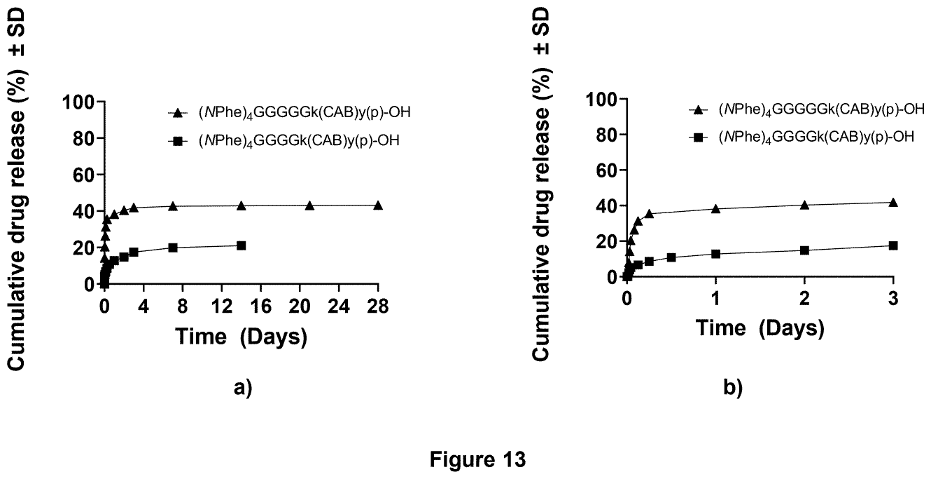

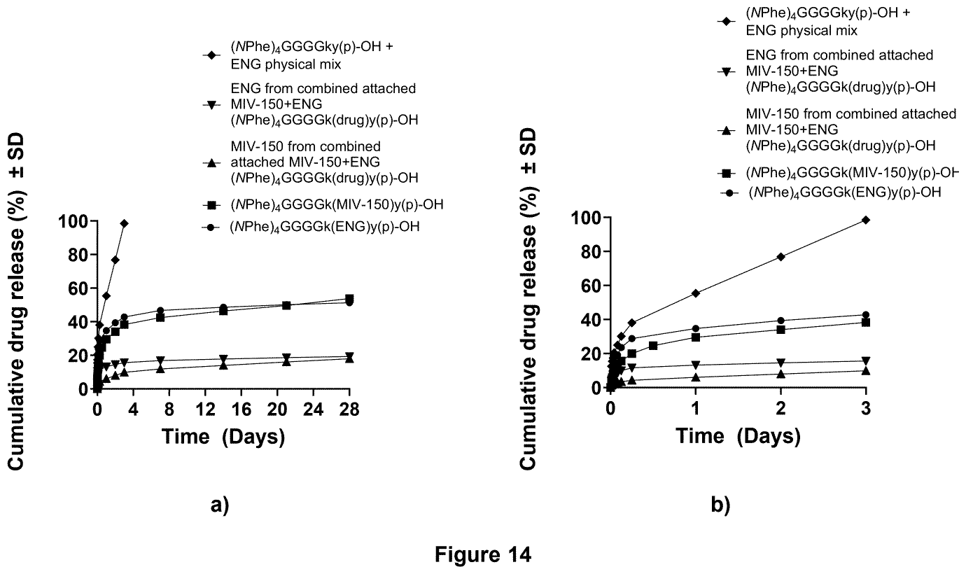

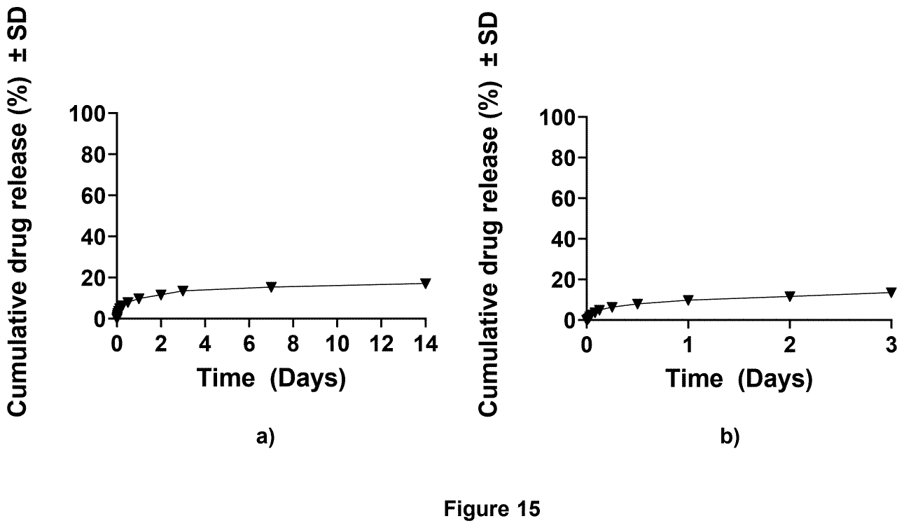

FIG. 12 demonstrates that the cumulative release of zidovudine is slower when zidovudine is chemically bonded to the compound of the present invention, when compared to a physical mix of peptide and zidovudine. Similar results were obtained for cabotegravir (FIG. 13), MIV-150 and etonogestrel (FIG. 14), and haloperidol (FIG. 15).

Natural peptides form the building blocks of proteins and tissues. They are composed of L-amino acids/L-peptides. Their use as a drug releasing hydrogel implant for administration under the skin is promising due to their high biocompatibility, but limited by their rapid degradation within hours by enzymes present in the human body. Embodiments of the present invention overcome stability issues by using peptide-mimetics, notably a combination of peptoids and D-peptides, which retain the positive properties of peptides (e.g. biocompatibility, easy drug attachment) with the ability to form hydrogels that will be stable for the duration of therapy.

The compound of the present invention can be tailored to degrade within the body by switching D-peptides with L-peptides and vice versa, over months into non-toxic components that are eliminated from the patient, meaning surgical removal of this implant will not be necessary.

The compound of the present invention possesses high chemical versatility (i.e. wide choice of chemical functional groups). Therefore multiple drugs can be attached directly to the peptide-mimetic hydrogel, formable from a solution of the compound, enabling large quantities of drug to be incorporated to meet in vivo therapeutic need for at least 28 days or the required dosage interval.

Drug detachment proceeds within seconds/minutes in physiological conditions after the hydrogel forms reducing potential for rapid burst release of drug upon injection. Drug release show the potential for sustained drug administration in a bid to minimise pharmacokinetic peaks and troughs in drug concentrations. The peptoid-peptides outlined herein according to the invention are purposefully small molecules that are cheap to manufacture, improving their potential to be clinically translated as a pharmaceutical product and effectively utilised within healthcare budgets for patient and societal benefit.

The concept of the present invention has been proven using the following drugs:
Zidovudine, MIV-150, cabotegravir, lamivudine (HIV/AIDS antiretrovirals)
Etonogestrel (contraceptive) both alone and combined with MIV-150
Haloperidol (antipsychotic)
Doxorubicin (cancer)

Rheological analysis demonstrated phosphatase enzyme instructed self-assembly, with hydrogels forming within minutes. Peptoid-peptides containing D-amino acids at lysine (k) and tyrosine positions (y) are particularly promising for long-acting delivery, displaying protease resistance. Drug release, via hydrolysis of drug-peptide linkage (e.g. ester), progresses under physiological conditions (37° C., pH 7.4, H₂O). Subcutaneous administration of peptoid-peptide with zidovudine covalently attached in Sprague Dawley rats demonstrated zidovudine blood plasma concentrations within the half maximal inhibitory concentration (IC₅₀) range (30-130 ng/ml) for 35 days.

The present invention overcomes the limitations outlined with existing products by using a superior in situ forming, enzyme-responsive, peptide-mimetic hydrogel drug delivery system. The use of such stimuli-responsive self-assembling peptide-mimetic systems as long-acting in situ sustained release implants has not been previously investigated.

Synthesis of drug-attached peptoid-peptides is outlined in FIG. 2. Collectively these motifs provide several advantages over existing drug delivery systems. Specifically, the implants will be administered as a soluble injection with low viscosity and volume, improving ease of administration for users by using narrower bore needles. As the invention exists as a soluble injection, it will not suffer from physical stability issues observed with suspensions.

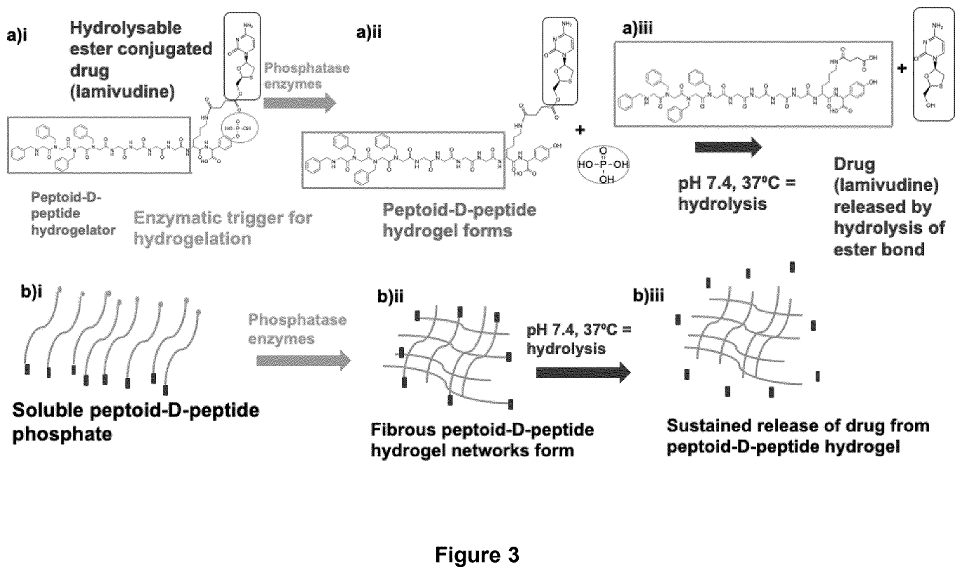

Following administration (concept outlined fully in FIG. 3), the hydrogel forms rapidly in response to phosphatase enzymes present within the hypodermis with fast sol-to-gel kinetics. Gel propensity was confirmed by a vial inversion assay whereby “gels” remain suspended. The fibrous architecture of the hydrogel was confirmed by SEM.

Alongside chemical attachment of drug to peptoid-peptide, hydrogel fibres provide a diffusional barrier to drug release, enabling sustained release properties and reducing burst release of drug. Gel formation can only truly be proven by rheological analysis. We have obtained promising data, observing hydrogel formation to occur within minutes exposure to phosphatase enzymes (Rheology data for each peptoid-peptide+drug FIGS. 4-10).

Drugs can be covalently attached to the hydrogel-forming molecule of the present invention from the outset. Peptide-mimetics provide a wide selection of chemical functional groups, enabling precise attachment of drugs directly to our hydrogel-forming molecule via a labile ester linkage. Drug release will be controlled by hydrolysis of this ester bond (see concept FIG. 3iii) reducing burst release of drug (FIG. 12) by 30-60% when chemically conjugated drug is compared to physically mixed controls. The peptide-mimetic motif can be altered to allow other bonds (e.g. amide) broke in physiological environments to be utilised, enabling attachment of a wide variety of drugs and control over their release rate.

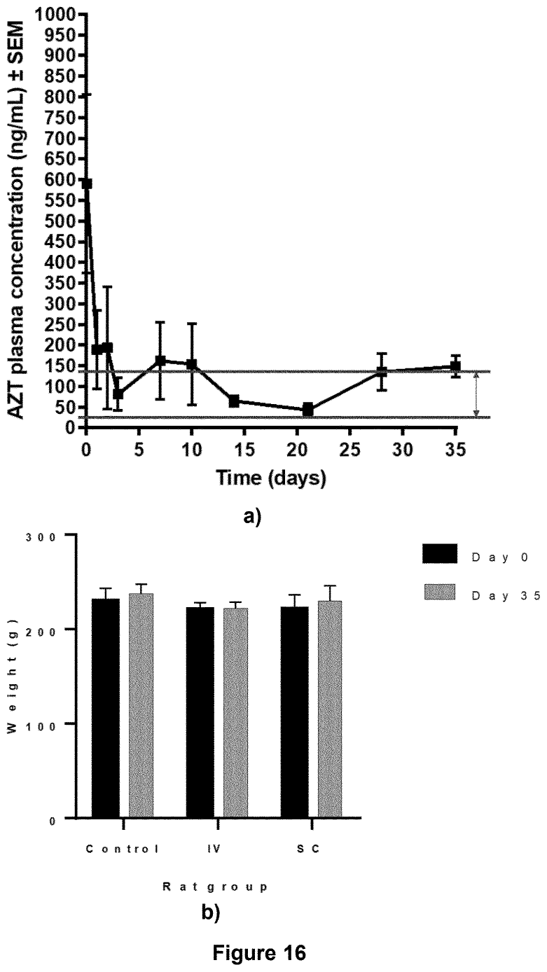

As FIG. 16 demonstrates, subcutaneous administration of D-peptide form of PP4G(AZT) in Sprague Dawley rats demonstrated zidovudine (AZT) blood plasma concentrations at least within the IC₅₀ range (30-130 ng/ml) of HIV for 35 days. No toxicity was demonstrated from preliminary results as weight of Sprague Dawley rats (n=6) at the beginning (day 0) and end (day 35) of the experiment in the control, were similar in intravenous and subcutaneous administration groups and negative controls (no peptoid-peptide or drug).

Unlike alternative stimuli-responsive in situ systems currently in research-which unhelpfully rely upon organic solvent exchange, photo-initiation, thermo-responsive polymers or pH-induced gelation—the system of the present invention makes use of endogenous enzymes as safer, more rapid and convenient mechanism for triggering gelation.

There is also a need for an in situ forming system that can deliver poorly water-soluble drugs with enhanced control over release rates. One way to increase solubility is to attach the drug directly to a water-soluble polymer. In one embodiment, the hydrogel forming peptide-mimetic compound of the present invention possess a single drug conjugatable group enabling precise drug attachment and improving drug solubility, enabling greater drug homogeneity and loading and allowing tuneable drug release rates from the peptide-based hydrogel (i.e. stimuli-responsive hydrogel formation and drug release/hydrolysis).

Peptide-based hydrogels are also more amenable to manipulation at the molecular scale relative to synthetic polymers offering the capability of tuning features such as drug release, viscoelastic and mechanical properties. Moreover, unlike conventional contraceptive implants, the peptide-based implants of the present invention biodegrade with time, eliminating the need for surgical removal. The compound of the present invention can be formed as a solid powder formulation to be dissolved in sterile water/buffer (e.g. water for injection) immediately prior to injection i.e. at the healthcare centre/clinic, which will reduce the effects of temperature fluctuations and lack of infrastructure within the developing world.

FIG. 15 demonstrates the L-peptide variant of peptoid-peptide PP4G and PP4G-MIV-150, where K (lysine) and Y (tyrosine) amino acid are L-enantiomers, breaks down within days of proteinase-K exposure. The D-peptide variant of peptoid-peptide PP4G, where k and y amino acid are unnatural D-enantiomers, remains significantly stable for 28 days exposure. Biostability can therefore be tailored, for example to break down over a particular dosage interval, in order to allow administration of next dose of PP4G+drug.

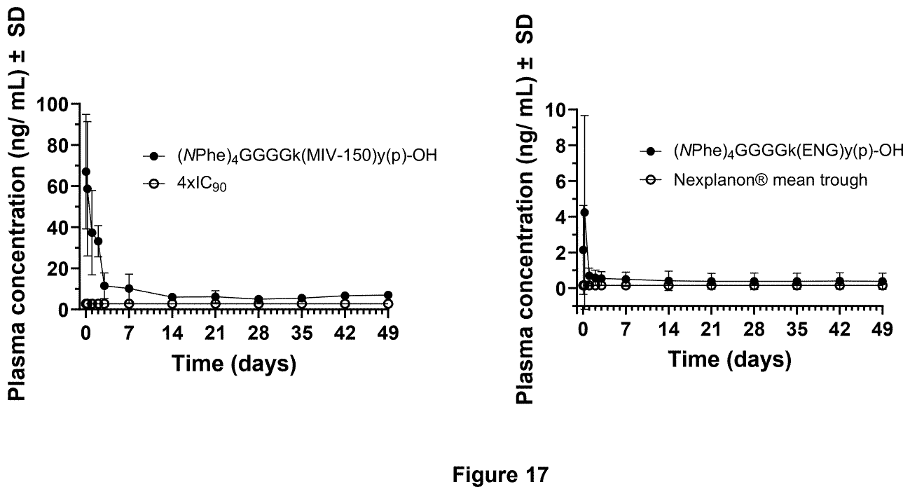

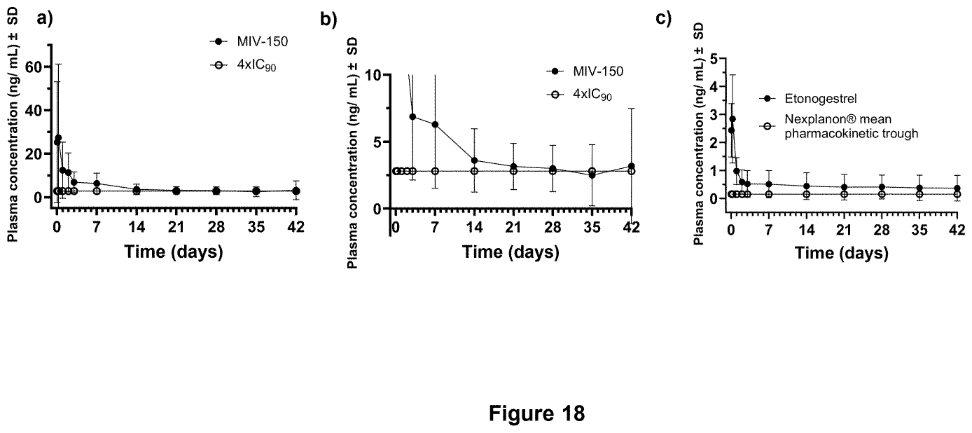

A drug attached to the peptide-mimetic molecule and administered to a recipient is detectable at least after 7, 14, 21, 28, 35, 42 or 49 days. FIGS. 17 and 18 show that a drug (in the examples, MIV-150 or etonogestrel) attached to the peptide-mimetic molecule of the present invention is detectable after administration even after 42 days (FIG. 18) or 49 days (FIG. 17). Data from FIG. 18 shows that multiple drugs can be delivered systemically within a single injectable product. Drug can be detected systemically to clinically relevant concentrations of MIV-150 (FIG. 18a, b) and contraceptive ENG (FIG. 18c) for 42 days when administered as a single injection using our platform.

In addition to the above, the presence of a phosphate group on the compound of the present invention ensures the product is in a soluble liquid form after addition of water, aiding ease of administration via injection to the skin site in patients. The presence of phosphatase enzymes within the skin, results in removal of the phosphate grouping reducing the overall solubility of the molecule and resulting in formation of a hydrogel (FIG. 3ii).

The physiological environment (pH 7.4, 37° C., H₂O) under the skin leads to hydrolysis of the bond between the drug and peptide-mimetic resulting in sustained release of drug from the hydrogel network i.e. reduced initial diffusion of drug (burst-release). Sustained drug release will minimise pharmacokinetic peaks and troughs in drug concentrations. Other advantages associated with peptide-mimetics are as follows:
Peptide-mimetics possess high chemical versatility compared to common polymers allowing precise drug conjugation and the ability to add specific enzyme cleavable groups to control hydrogel formation.
Peptide-mimetics and their biodegradation products will act as structural mimetics of peptides and will be biocompatible.
Drugs are conjugated directly to the peptide-mimetic hydrogel motif thus overcoming limitations with existing long-acting injectable preparations, namely low water solubility, which limits drug loading/therapeutic efficacy.
Peptide-mimetics will be more stable than their corresponding peptides to protease degradation and will retain its hydrogel matrix and release properties for longer (extended half-life).

Unless defined otherwise, all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

Unless otherwise indicated, all numbers expressing quantities of ingredients, properties such as size, weight, reaction conditions and so forth used in the specification and claims are to the understood as being modified in all instances by the term “about”. Accordingly, unless indicated to the contrary, the numerical parameters set forth in the following specification and attached claims are approximations that may vary depending upon the desired properties sought to be obtained by the present subject matter. At the very least, and not as an attempt to limit the application of the doctrine of equivalents to the scope of the claims, each numerical parameter should at least be construed in light of the number of reported significant digits and by applying ordinary rounding techniques.

Throughout the application, descriptions of various embodiments use “comprising” language; however, it will be understood by one of skill in the art, that in some instances, an embodiment can alternatively be described using the language “consisting essentially of or “consisting of.”

1. A compound formed at least partially from a peptide-mimetic molecule, wherein:
the compound is configured for the attachment of one or more drug molecules;
the compound is further configured to release the one or more drug molecules under physiological conditions;
a solution of the compound forms a hydrogel in response to phosphatase;
and wherein the compound comprises: a peptide-mimetic backbone formed from one or more of the group comprising peptoids, D-peptides, or β-homo peptides; a phosphate group which when cleaved drives formation of a hydrogel; and, a site for covalent attachment of a drug.

2. The compound of claim 1, wherein the site for covalent attachment of a drug is provided by an amino acid that has a primary amine (—NH₂) as its functional group that allows drug attachment.

3. The compound of claim 2, wherein the site for covalent attachment of a drug is an ε-amino group of lysine, or an L- or D-cysteine.

4. The compound of claim 1, wherein the one or more drugs are releasable from the compound by hydrolysis.

5. The compound of claim 1, wherein the one or more drugs are attached to the compound by an ester, carbamate, amide, ether, disulphide or hydrazone linkage.

6. The compound of claim 1, the drug attachable to the compound is an HIV/AIDS retroviral drug, contraceptive, antipsychotic, or cancer treatment drug.

7. The compound of claim 6, wherein the drug is zidovudine, MIV-150, cabotegravir, lamivudine, etonogestrel, haloperidol, or doxorubicin.

8. The compound of claim 1, wherein the compound comprises L-peptides.

9. The compound of claim 1, having the formula:

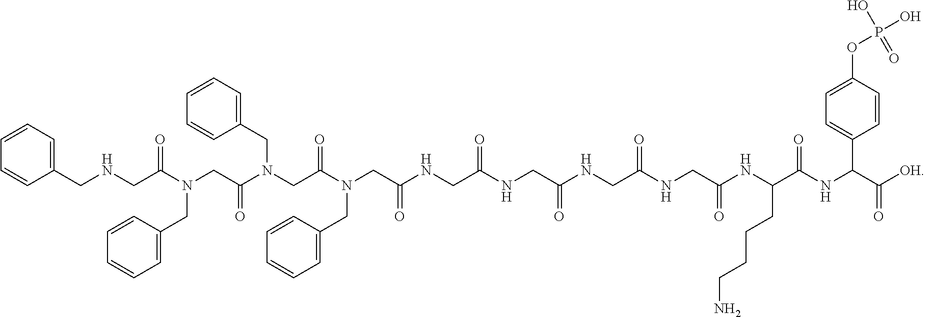 [SMILES: NCCCCC(NC(=O)CNC(=O)CNC(=O)CNC(=O)CNC(=O)CN(Cc1ccccc1)C(=O)CN(Cc1ccccc1)C(=O)CN(Cc1ccccc1)C(=O)CNCc1ccccc1)C(=O)NC(C(=O)O)c1ccc(OP(=O)(O)O)cc1]

10. The compound of claim 1 attached to a drug for use as a medicament.

11. The compound of claim 10 for use in the prevention or treatment of AIDS, psychiatric illnesses, cancer, or as a contraceptive.

12. The compound of claim 1, wherein at least 10% of a concentration of the compound, with or without a drug attached thereto, remains after 28 days of incubation with proteinase K.

13. An injectable delivery system for long-acting administration of drugs, the system comprising a solution, the solution comprising the compound of claim 1 with a drug attached thereto, wherein the solution can be injected under the skin, and wherein upon injection and exposure to enzymes within or under the skin, the compound forms a hydrogel.

14. The injectable delivery system of claim 13 comprising the compound having a first drug attached thereto, and the compound having a second drug attached thereto, wherein the second drug is chemically distinct from the first drug.

15. The injectable delivery system of claim 13 for use as a medicament.

16. The injectable delivery system of 15 for use in the prevention or treatment of AIDS, depression, cancer, or as a contraceptive.

17. The injectable delivery system of claim 13, wherein the drug is detectable in the recipient after at least 28 days.

18. The injectable delivery system of claim 17, wherein the drug is detectable in the recipient after at least 49 days.
